(19) United States
(12) Patent Application Publication
(10) Pub. No.: US 2023/0122622 A1
(43) Pub. Date: Apr. 20, 2023

(54) Composition Containing Polyamideimide Precursor or Polyamideimide, and Polyamideimide Film Produced Using the Same

(71) Applicants: SK Innovation Co., Ltd., Seoul (KR); SK ie technology Co., Ltd., Seoul (KR)

(72) Inventors: Jin Hyung Park, Daejeon (KR); Hye Ri Kim, Daejeon (KR); Seung Min Jeon, Daejeon (KR)

(21) Appl. No.: 17/966,935

(22) Filed: Oct. 17, 2022

(30) Foreign Application Priority Data
Oct. 18, 2021 (KR) 10-2021-0138243

(51) Int. Cl. C08L 79/08; C07C 233/81; C08J 5/18; C07C 211/37; C07D 493/04

(52) U.S. Cl. CPC C08L 79/08; C07C 233/81; C08J 5/18; C07C 211/37; C07D 493/04; C08J 2379/08; C08L 2203/16

(57) ABSTRACT

The present disclosure relates to a composition containing a polyamideimide precursor and/or polyamideimide, a polyamideimide film produced using the composition, and a display device including the polyamideimide film. The polyamideimide film produced using the composition and the display device including the polyamideimide film have excellent mechanical and optical properties.

### CROSS-REFERENCE TO RELATED APPLICATION

This application claims priority to Korean Patent Application No. 10-2021-0138243, filed Oct. 18, 2021, the disclosure of which is hereby incorporated by reference in its entirety.

### BACKGROUND OF THE INVENTION

### Field of the Invention

The following disclosure relates to a composition containing a polyamideimide precursor or polyamideimide, and a polyamideimide film produced using the same.

### Description of Related Art

In general, a polyimide-based resin has excellent mechanical and thermal properties, and thus has been applied to various fields such as a field of an insulating substrate for forming a circuit and a device. Recently, research and development have been conducted to replace cover glass for a display device with a polymer material by using these properties.

The main physical properties required for using the polyimide-based resin in a display device are optical and mechanical properties. As a method for preparing polyimide having excellent optical and mechanical properties, a method of copolymerizing a dicarbonyl compound with transparent polyimide and increasing a ratio of a monomer having strong straightness and rigidity to the dicarbonyl compound in order to improve mechanical strength of the polyimide at this time has been reported. However, when an introduction ratio of the monomer having strong straightness and rigidity is high, a level of difficulty of a process is increased or the process is impossible due to deterioration of handleability of the solution, and an intermolecular space becomes dense, which causes an increase in formation of a charge transfer complex (CTC) in the polyimide, resulting in deterioration of optical properties. When the charge transfer complex is formed, the polyimide is colored brown or yellow, and thus a transmittance in the visible light region is reduced, which makes it difficult to apply the polyimide to a material for a display device. In addition, the introduction of the monomer having strong rigidity accelerates cloudiness in a drying process at a high temperature, which causes deterioration of optical properties. Therefore, it is required to lower the temperature in the drying process, and as a result, sufficient productivity is not secured or a level of difficulty of the process application is increased.

In addition, a method of suppressing formation of an intramolecular charge transfer complex using an alicyclic diamine or an aliphatic diamine as a diamine component is known as a method for making polyimide colorless and transparent. Japanese Patent Laid-Open Publication No. 2002-161136 discloses polyimide obtained by imidizing a polyimide precursor formed of an aromatic acid dianhydride such as pyromellitic dianhydride and trans-1,4-diaminocyclohexane, but although polyimide exhibits high transparency, there is a problem in that mechanical properties are deteriorated.

In addition, although attempts have been made to use various functional monomers as a method for converting the yellow color of polyimide to colorless and transparent, it is difficult to access the method due to problems in the manufacturing process, such as a rapid increase in viscosity during polymerization or difficulty in purification, and it is insufficient to prevent the deterioration of excellent mechanical properties inherent in polyimide in spite of securing transparency.

Therefore, there is a need to develop a technology for polyimide that has excellent optical properties and prevents deterioration of intrinsic excellent mechanical properties so as to be applicable to various display device material fields comprising cover glass replacement materials, and in particular, implements a high modulus so as to be applicable to a wider range.

### SUMMARY OF THE INVENTION

An embodiment of the present disclosure is directed to providing a composition containing a polyamideimide precursor or polyamideimide that may implement further improved mechanical and optical properties of a film in comparison to polyamideimide according to the related art.

Another embodiment of the present disclosure is directed to providing a polyamideimide film comprising a cured product of the composition and a display device comprising the polyamideimide film.

In one general aspect, a composition contains: a polyamideimide precursor or polyamideimide having a unit derived from an aromatic diamine, a unit derived from an aromatic dianhydride, and a unit derived from an aromatic diacid dichloride; and a solvent,

wherein the aromatic diamine comprises a compound represented by the following Chemical Formula 1 (hereinafter, referred to as AB-TFMB), the aromatic dianhydride comprises 9,9-bis(3,4-dicarboxyphenyl)fluorene dianhydride (BPAF), and the aromatic diacid dichloride comprises terephthaloyl dichloride (TPC):

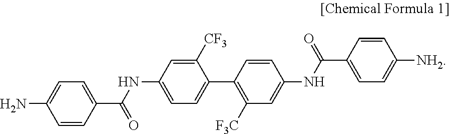 [SMILES: Nc1ccc(C(=O)Nc2ccc(-c3ccc(NC(=O)c4ccc(N)cc4)cc3C(F)(F)F)c(C(F)(F)F)c2)cc1]

In another general aspect, a polyamideimide film comprises a cured product of the composition. In still another general aspect, a display device comprises the polyamideimide film.

Other features and aspects will be apparent from the following detailed description and the claims.

### DESCRIPTION OF THE INVENTION

Embodiments disclosed in the present specification may be modified into many different forms and the technology according to one embodiment is not limited to the embodiments described below. Furthermore, in the entire specification, unless explicitly described otherwise, “comprising” any components will be understood to imply the inclusion of other components but not the exclusion of any other components.

In addition, unless defined otherwise, the technical terms and scientific terms used in the present specification have the same meanings as commonly understood by those skilled in the art disclosed in the present specification.

A numerical range used in the present specification comprises upper and lower limits and all values within these limits, increments logically derived from a form and span of a defined range, all double limited values, and all possible combinations of the upper and lower limits in the numerical range defined in different forms. As an example, when a content of a composition is limited to 10% to 80% or 20% to 50%, a numerical range of 10% to 50% or 50% to 80% should also be interpreted as described in the present specification. Unless otherwise specifically defined in the present specification, values out of the numerical ranges that may occur due to experimental errors or rounded values also fall within the defined numerical ranges.

Hereinafter, unless otherwise specifically defined in the present specification, a “combination thereof” may refer to mixing or copolymerization of constituents.

Hereinafter, unless otherwise specifically defined in the present specification, “A and/or B” may refer to an aspect comprising both A and B, and may refer to an aspect selected from A and B.

Hereinafter, unless otherwise specifically defined in the present specification, the term “polymer” may refer to a molecule having a relatively high molecular weight, and a structure thereof may comprise multiple repeats of units derived from a molecule having a low molecular weight. In one embodiment, the polymer may be an alternating copolymer, a block copolymer, a random copolymer, a graft copolymer, a gradient copolymer, a branched copolymer, a crosslinked copolymer, or a copolymer comprising all of these copolymers (for example, a polymer containing more than one kind of monomers). In another embodiment, the polymer may be a homopolymer (for example, a polymer containing one kind of monomers).

Hereinafter, unless otherwise specifically defined in the present specification, the term “polyamic acid” may refer to a polymer having a structural unit having an amic acid moiety, and the term “polyamideimide” may refer to a polymer having a structural unit having an amide moiety and an imide moiety.

Hereinafter, unless otherwise specifically defined in the present specification, a polyamideimide film may be a film containing polyamideimide, and specifically, a high heat-resistant film produced by subjecting a dianhydride compound and a diacid dichloride to solution polymerization in a diamine compound solution to prepare polyamic acid, and then imidizing the polyamic acid.

Hereinafter, unless otherwise specifically defined in the present specification, it will be understood that when an element such as a layer, a film, a thin film, a region, a plate, or the like, is referred to as being “above” or “on” another element, it may be “directly on” another element or may have an intervening element present therebetween.

Hereinafter, unless otherwise specifically defined in the present specification, the term “substituted” means that a hydrogen atom in a compound is substituted with a substituent. For example, the substituent may be selected from deuterium, a halogen atom (F, Br, Cl, or I), a hydroxy group, a nitro group, a cyano group, an amino group, an azido group, an amidino group, a hydrazino group, a hydrazono group, a carbonyl group, a carbamyl group, a thiol group, an ester group, a carboxyl group or a salt thereof, a sulfonic acid group or a salt thereof, a phosphoric acid or a salt thereof, a C₁₋₃₀ alkyl group, a C₂₋₃₀ alkenyl group, a C₂₋₃₀ alkynyl group, a C₆₋₃₀ aryl group, a C₇₋₃₀ arylalkyl group, a C₁₋₃₀ alkoxy group, a C₁₋₂₀ heteroalkyl group, a C₃₋₂₀ heteroarylalkyl group, a C₃₋₃₀ cycloalkyl group, a C₃₋₁₅ cycloalkenyl group, a C₆₋₁₅ cycloalkynyl group, a C₂₋₃₀ heterocyclic group, and a combination thereof.

Hereinafter, a composition according to one embodiment will be described.

The composition according to one embodiment is a composition containing: a polyamideimide precursor and/or polyamideimide having a unit derived from an aromatic diamine, a unit derived from an aromatic dianhydride, and a unit derived from an aromatic diacid dichloride; and a solvent,

wherein the aromatic diamine may comprise a compound represented by the following Chemical Formula 1 (AB-TFMB), the aromatic dianhydride may comprise 9,9-bis(3,4-dicarboxyphenyl)fluorene dianhydride (BPAF), and the aromatic diacid dichloride may comprise terephthaloyl dichloride (TPC):

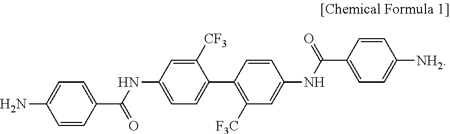 [SMILES: Nc1ccc(C(=O)Nc2ccc(-c3ccc(NC(=O)c4ccc(N)cc4)cc3C(F)(F)F)c(C(F)(F)F)c2)cc1]

In one embodiment, the composition may be a composition for forming a polyamideimide film.

As an example, AB-TFMB which is an aromatic diamine, TPC which is an aromatic diacid dichloride, and BPAF which is an aromatic dianhydride containing fluorene having a sterically bulky structure are used in combination, such that a polyamideimide film excellent in both mechanical properties and optical properties may be provided. Specifically, in a case of using a combination in which AB-TFMB and TPC are used in the same manner as described above, but 4,4′-(hexafluoroisopropylidene)diphthalic anhydride (hereinafter, referred to as 6FDA) is used instead of BPAF containing fluorene having a sterically bulky structure, cloudiness is accelerated in a drying process at a high temperature, and thus transparency of the film may not be secured. On the other hand, the composition according to one embodiment contains all of AB-TFMB, BPAF, and TPC, such that it is possible to provide a polyamideimide film having a significantly excellent transmittance and transparency, having excellent optical properties due to a low yellow index, and having significantly excellent mechanical strength, even when dried at a high temperature.

In this case, while not wishing to be bound by a certain theory, AB-TFMB may comprise a plurality of amide bonds in the molecule, and an amide group may be further induced according to the reaction between AB-TFMB and TPC. Therefore, a plurality of amide bonds are comprised in a polyamideimide resin prepared using the composition, such that an intramolecular interaction and/or an intermolecular interaction of the amide bonds may be increased, and thus mechanical properties of the polyamideimide resin may be significantly improved.

In addition, while not wishing to be bound by a certain theory, AB-TFMB, TPC, and BPAF all contain an aromatic ring, such that a content of carbon in the polyamideimide resin may be increased. Therefore, it is possible to provide a film having more excellent mechanical properties and sufficient optical properties.

As the aromatic diamine, AB-TFMB may be used alone, or a mixture of aromatic diamines commonly used in the art may be used, if necessary. For example, in addition to AB-TFMB, the composition may further contain a second aromatic diamine different from AB-TFMB. The second aromatic diamine may contain, for example, a substituted or unsubstituted C₆₋₃₀ aromatic ring, and in this case, the aromatic ring may be a single ring, a fused ring in which two or more aromatic rings are fused, or an unfused ring in which two or more aromatic rings are connected by a single bond, a Cis alkylene group, O, or C(═O). In addition, a fluorine substituent may be introduced into the second aromatic diamine, and the use of the aromatic diamine into which a fluorine substituent is introduced may help improve optical properties of the resin prepared using the composition according to one embodiment. Specifically, the second aromatic diamine may contain an aromatic ring substituted with one or two or more trifluoroalkyl groups, and the aromatic ring substituted with the trifluoroalkyl group may be unsubstituted or further substituted with a substituent other than the trifluoroalkyl group.

As a specific example, the second aromatic diamine may be, but is not limited to, one or more aromatic diamines selected from the group consisting of 2,2′-bis(trifluoromethyl)-benzidine (TFMB), 2,2-bis[4-(4-aminophenoxy)phenyl]hexafluoropropane (4BDAF), 2,2-bis(3-amino-4-hydroxyphenyl)hexafluoropropane (6FAP), 3,4′-oxydianiline (ODA), p-phenylenediamine (pPDA), m-phenylenediamine (mPDA), p-methylenedianiline (pMDA), m-methylenedianiline (mMDA), 1,3-bis(3-aminophenoxy)benzene (133APB), 1,3-bis(4-aminophenoxy)benzene (134APB), 1,4-bis(4-aminophenoxy)benzene (144APB), bis(4-aminophenyl)sulfone (ODDS), bis(3-aminophenyl)sulfone (3DDS), 2,2-bis[4-(4-aminophenoxy)phenyl]propane (6HMDA), and derivatives thereof, or a mixture thereof. Specifically, the second aromatic diamine may comprise 2,2′-bis(trifluoromethyl)-benzidine (TFMB).

As the aromatic diamine, AB-TFMB may be used alone, or a mixture of AB-TFMB and the second aromatic diamine may be used. In the case where the mixture of AB-TFMB and the second aromatic diamine is used, a mixing ratio of them is not limited, and AB-TFMB (the unit derived from AB-TFMB) may be contained in an amount of 1 mol % or more, 2 mol % or more, 5 mol % or more, 10 mol % or more, 20 mol % or more, 30 mol % or more, 40 mol % or more, 50 mol % or more, 55 mol % or more, 60 mol % or more, or 70 mol % or more, with respect to the total number of moles of the aromatic diamine (the unit derived from the aromatic diamine), and may be contained in an amount of 99 mol % or less, 98 mol % or less, 95 mol % or less, 90 mol % or less, 80 mol % or less, 75 mol % or less, 70 mol % or less, 65 mol % or less, 60 mol % or less, or 50 mol % or less, with respect to the total number of moles of the aromatic diamine, but the amount of AB-TFMB is not limited thereto.

The polyamideimide precursor and/or polyamideimide may further have a unit derived from an aliphatic ring-containing dianhydride. As the aliphatic ring-containing dianhydride, an aliphatic ring-containing dianhydride commonly used in the art may be used, but is not limited thereto. The aliphatic ring-containing dianhydride may refer to a dianhydride containing at least one aliphatic ring, and the aliphatic ring may be a single ring, a fused ring in which two or more aliphatic rings are fused, or an unfused ring in which two or more aliphatic rings are connected by a single bond, a substituted or unsubstituted Cis alkylene group, O, or C(═O). For example, the aliphatic ring-containing dianhydride may be one or more aliphatic ring-containing dianhydrides selected from the group consisting of cyclobutane-1,2,3,4-tetracarboxylic dianhydride (CBDA), cyclopentane-1,2,3,4-tetracarboxylic dianhydride (CPDA), cyclohexane-1,2,3,4-tetracarboxylic dianhydride (CHDA), 5-(2,5-dioxotetrahydrofuryl)-3-methyl-cyclohexene-1,2-dicarboxylic dianhydride (DOCDA), bicyclo[2.2.2]-7-octene-2,3,5,6-tetracarboxylic dianhydride (BTA), 1,2,4-tricarboxy-3-methylcarboxy cyclopentane dianhydride, 1,3-dimethyl-1,2,3,4-cyclobutanetetracarboxylic dianhydride (DM-CBDA), 1,3-diethyl-1,2,3,4-cyclobutanetetracarboxylic dianhydride (DE-CBDA), 1,3-diphenyl-1,2,3,4-cyclobutanetetracarboxylic dianhydride (DPh-CBDA), 4-(2,5-dioxotetrahydrofuran-3-yl)-1,2,3,4-tetrahydronaphthalene-1,2-dicarboxylic dianhydride (TDA), and derivatives thereof, or a mixture thereof. More specifically, the aliphatic ring-containing dianhydride may comprise cyclobutane-1,2,3,4-tetracarboxylic dianhydride (CBDA).

In the case where the aliphatic ring-containing dianhydride is further contained, a mixing ratio thereof is not limited, and the aliphatic ring-containing dianhydride (the unit derived from the aliphatic ring-containing dianhydride) may be contained in an amount of 1 mol % or more, 2 mol % or more, 5 mol % or more, 10 mol % or more, 20 mol % or more, 30 mol % or more, 40 mol % or more, 50 mol % or more, 55 mol % or more, 60 mol % or more, or 70 mol % or more, with respect to the total number of moles of the aromatic diacid dichloride (the unit derived from the aromatic diacid dichloride), the aromatic dichloride (the unit derived from the aromatic dichloride), and the aliphatic ring-containing dianhydride (the unit derived from the aliphatic ring-containing dianhydride), and may be contained in an amount of 99 mol % or less, 98 mol % or less, 95 mol % or less, 90 mol % or less, 80 mol % or less, 75 mol % or less, 70 mol % or less, 65 mol % or less, 60 mol % or less, or 50 mol % or less, with respect to the total number of moles of the aromatic diacid dichloride (the unit derived from the aromatic diacid dichloride), the aromatic dichloride (the unit derived from the aromatic dichloride), and the aliphatic ring-containing dianhydride (the unit derived from the aliphatic ring-containing dianhydride). However, the amount of the aliphatic ring-containing dianhydride is not limited to the above range.

As the aromatic diacid dichloride, TPC may be used alone, or a mixture of TPC and an aromatic diacid dichloride commonly used in the art may be used. For example, one or more selected from the group consisting of isophthaloyl dichloride (IPC), 1,1′-biphenyl-4,4′-dicarbonyl dichloride (BPC), 1,4-naphthalenedicarboxylic dichloride (NPC), 2,6-naphthalenedicarboxylic dichloride (NTC), 1,5-naphthalenedicarboxylic dichloride (NEC), 4,4′-oxybis(benzoyl chloride) (DEDC), and derivatives thereof, or a mixture thereof may be used, but is not limited thereto. In a case where an amide structure is formed in the polymer chain by the aromatic diacid dichloride, optical properties, and in particular, mechanical strength such as a modulus of a film may be further improved.

TPC (the unit derived from TPC) may be contained in the composition according to one embodiment in an amount of 1 mol % or more, 2 mol % or more, 5 mol % or more, 10 mol % or more, 20 mol % or more, 30 mol % or more, 40 mol % or more, 50 mol % or more, 55 mol % or more, 60 mol % or more, or 70 mol % or more, with respect to the total number of moles of the aromatic dianhydride (the unit derived from the aromatic dianhydride) and the aromatic diacid dichloride (the unit derived from the aromatic diacid dichloride), and may be contained in an amount of 99 mol % or less, 95 mol % or less, 90 mol % or less, 80 mol % or less, 75 mol % or less, or 70 mol % or less, with respect to the total number of moles of the aromatic dianhydride (the unit derived from the aromatic dianhydride) and the aromatic diacid dichloride (the unit derived from the aromatic diacid dichloride), but the amount of TPC is not limited thereto. The composition according to one embodiment contains TPC, such that mechanical properties such as a modulus of a polyamideimide film are further improved, and an increase in retardation in a thickness direction or deterioration of optical properties may be reduced. Therefore, even when the composition is applied to a display device or the like, a screen distortion may be reduced.

In one embodiment, BPAF contains fluorene having a sterically bulky structure, such that a precursor composition having strong rigidity may suppress deterioration of optical properties (cloudiness) even in a high-temperature process. In this case, BPAF may be used alone, or a mixture of aromatic dianhydrides commonly used in the art may be used. In this case, the aromatic dianhydride may refer to a dianhydride containing at least one aromatic ring, and the aromatic ring may be a single ring, a fused ring in which two or more aromatic rings are fused, or an unfused ring in which two or more aromatic rings are connected by a single bond, a substituted or unsubstituted C₁₋₅ alkylene group, O, or C(═O). For example, the aromatic dianhydride may be one or more aromatic ring-containing dianhydrides selected from the group consisting of biphenyltetracarboxylic dianhydride (BPDA), 4,4′-oxydiphthalic dianhydride (ODPA), sulfonyl diphthalic anhydride (SO2DPA), (isopropylidenediphenoxy)bis(phthalic anhydride) (6HDBA), 4-(2,5-dioxotetrahydrofuran-3-yl)-1,2,3,4-tetrahydronaphthalene-1,2-dicarboxylic dianhydride (TDA), pyromellitic dianhydride (PMDA), benzophenone-3,3′,4,4′-tetracarboxylic dianhydride (BTDA), bis(carboxyphenyl) dimethyl silane dianhydride (SiDA), bis(dicarboxyphenoxy) diphenyl sulfide dianhydride (BDSDA), and derivatives thereof, or a mixture thereof.

BPAF (the unit derived from BPAF) may be contained in the composition according to one embodiment in an amount of 1 mol % or more, 2 mol % or more, 5 mol % or more, 10 mol % or more, 15 mol % or more, 20 mol % or more, 30 mol % or more, 40 mol % or more, 50 mol % or more, 55 mol % or more, 60 mol % or more, or 70 mol % or more, with respect to the total number of moles of the aromatic dianhydride (the unit derived from the aromatic dianhydride) and the aromatic diacid dichloride (the unit derived from the aromatic diacid dichloride), and may be contained in an amount of 99 mol % or less, 95 mol % or less, 90 mol % or less, 80 mol % or less, 75 mol % or less, 70 mol % or less, 60 mol % or less, 50 mol % or less, 45 mol % or less, or 40 mol % or less, with respect to the total number of moles of the aromatic dianhydride (the unit derived from the aromatic dianhydride) and the aromatic diacid dichloride (the unit derived from the aromatic diacid dichloride), but the amount of BPAF is not limited thereto.

In a case where the aliphatic ring-containing dianhydride is further contained in the composition according to one embodiment, a ratio of the number of moles of the aliphatic ring-containing dianhydride (the unit derived from the aliphatic ring-containing dianhydride) to the total number of moles of the aromatic diacid dichloride (the unit derived from the aromatic diacid dichloride) and the aromatic dianhydride (the unit derived from the aromatic dianhydride) may be 99:1 to 1:99, 95:5 to 10:90, 95:5 to 30:70, 95:5 to 40:60, 90:10 to 30:70, 80:20 to 30:70, 80:20 to 45:55, 80:20 to 40:60, or 75:25 to 45:65, but is not limited thereto. In this case, the molar ratio may be a molar ratio when the number of moles of the aromatic diamine (the unit derived from the aromatic diamine) is 100.

The above content ranges are examples exemplified in order to satisfy the desired physical properties, and may be changed according to the composition of the monomer, but are not limited thereto.

The composition may further contain a commonly used solvent. The solvent may be an organic solvent, specifically, a polar solvent, and more specifically, one or two or more selected from the group consisting of N,N-dimethylacetamide (DMAc), N-methyl-2-pyrrolidone (NMP), dimethylformamide (DMF), methyl sulfoxide (DMSO), ethyl cellosolve, methyl cellosolve, acetone, ethyl acetate, m-cresol, gamma-butyrolactone (GBL), and derivatives thereof, but is not limited thereto.

The composition may further contain an additive, if necessary, in addition to the components described above. The additive may be added to improve film formation, adhesion, optical properties, mechanical properties, flame retardancy, and the like, and may be, for example, a flame retardant, an adhesion enhancer, inorganic particles, an antioxidant, an ultraviolet inhibitor, and/or a plasticizer, but is not limited thereto.

The composition may be prepared by a method of adding all monomers containing an aromatic diamine, an aromatic dianhydride, and an aromatic diacid dichloride and then precipitating the monomers, and may be prepared by a method of mixing an aromatic diamine and an aromatic diacid dichloride, precipitating the mixture, and then mixing the other monomers.

In another embodiment, there is provided a polyamideimide film comprising a cured product of the composition according to one embodiment.

In this case, the polyamideimide film according to one embodiment may have a transmittance of 87.0% or more, 87.5% or more, 88.0% or more, 88.3% or more, 88.5% or more, 89.0% or more, 89.5% or more, or 90.0% or more, when measured at 400 nm to 700 nm according to ASTM D1003 standard, but is not limited thereto.

In addition, the polyamideimide film according to one embodiment may have a haze of 2.0% or less, 1.5% or less, 1.3% or less, 1.2% or less, 1.0% or less, 0.9% or less, 0.8% or less, 0.7% or less, 0.6% or less, 0.5% or less, or 0.4% or less, when measured according to ASTM D1003 standard, but is not limited thereto.

In addition, the polyamideimide film according to one embodiment may have a yellow index of 7.0 or less, 6.5 or less, 6.0 or less, 5.8 or less, 5.5 or less, 5.0 or less, 4.5 or less, or 4.0 or less, when measured according to ASTM E313 standard, but is not limited thereto.

In addition, the polyamideimide film according to one embodiment may have a modulus of 6.0 GPa or more, 6.5 GPa or more, 6.8 GPa or more, 7.0 GPa or more, 7.5 GPa or more, 8.0 GPa or more, or 9.5 GPa or more, when measured according to ASTM D882 standard.

The physical property values of the polyamideimide film may be values derived by measuring the polyamideimide film subjected to a drying process at 80° C. to 100° C., 85° C. to 95° C., or about 90° C., or may be values derived by measuring the polyamideimide film subjected to a drying process at a high temperature of 110° C. to 160° C., 120° C. to 160° C., 130° C. to 150° C., or about 140° C.

The polyamideimide film according to one embodiment is produced using the composition containing a polyamideimide precursor and/or polyamideimide having a combination of a unit derived from AB-TFMB, a unit derived from BPAF, and a unit derived from TPC according to one embodiment, such that a film simultaneously satisfying a transmittance of 87.0% or more, a haze of 2.0% or less, a yellow index of 7.0 or less, and a modulus of 6.0 GPa or more may be provided as desired.

A thickness of the polyamideimide film may be 1 μm to 500 μm, 10 μm to 250 μm, 10 μm to 100 μm, 20 μm to 100 μm, or 20 μm to 80 μm, but is not limited thereto.

The polyamideimide film according to one embodiment may be prepared by applying the composition containing a polyamideimide precursor and/or polyamideimide and a solvent according to one embodiment to a substrate, and then performing drying and/or stretching, or may be prepared by a solution casting method.

More specifically, the polyamideimide film according to one embodiment may be prepared by preparing a polyamideimide resin by imidizing a polyamideimide precursor and/or polyamideimide and a solvent, and forming a film by applying a resin composition in which the polyamideimide resin is dissolved in an organic solvent to a substrate.

The organic solvent may be one or two or more polar solvents selected from the group consisting of dimethylacetamide (DMAc), N-methyl-2-pyrrolidone (NMP), dimethylformamide (DMF), methyl sulfoxide (DMSO), ethyl cellosolve, methyl cellosolve, acetone, ethyl acetate, m-cresol, gamma-butyrolactone (GBL), and derivatives thereof, but is not limited thereto.

The imidization may be performed through chemical imidization using one or two or more selected from an imidization catalyst and a dehydrating agent. One or two or more selected from pyridine, isoquinoline, and β-quinoline may be used as the imidization catalyst. In addition, one or two or more selected from acetic anhydride, phthalic anhydride, and maleic anhydride may be used as the dehydrating agent. However, an imidization catalyst and a dehydrating agent commonly used may be selected and used as the imidization catalyst and the dehydrating agent, but the imidization catalyst and the dehydrating agent are not limited to the above types.

In addition, in the preparing of the polyamideimide resin, a polyamideimide resin may be prepared by mixing the polyamic acid solution with an additive such as a flame retardant, an adhesion enhancer, inorganic particles, an antioxidant, an ultraviolet inhibitor, or a plasticizer. In addition, after the imidization is performed, the resin may be purified using a solvent to obtain a solid, and the solid may be dissolved in a solvent, thereby obtaining a polyamideimide resin composition. In this case, the solvent may comprise, for example, DMAc, but is not limited thereto.

The forming of the film is a process of forming a film by applying the polyamideimide resin composition to a substrate and then performing a heat treatment. For example, glass, stainless steel, a film, or the like may be used as the substrate, and the application may be performed by a die coater, an air knife, a reverse roll, spray, a blade, casting, gravure, spin coating, or the like.

The heat treatment may be performed, for example, in stages. For example, a stepwise heat treatment may be performed by performing primary drying at 70° C. to 160° C. for 1 minute to 2 hours and performing secondary drying at 150° C. to 450° C. for 1 minute to 2 hours. However, the heat treatment is not limited to the temperature and time conditions, and for example, the primary drying may be performed at 25° C. to 220° C., 80° C. to 150° C., 70° C. to 110° C., 130° C. to 150° C., 90° C., 120° C., or 140° C. for 1 minute to 300 minutes, 10 minutes to 150 minutes, 10 minutes to 90 minutes, 20 minutes to 60 minutes, or 30 minutes, and the secondary drying may be performed at 200° C. to 500° C., 200° C. to 300° C., 220° C. to 300° C., or 250° C. to 300° C. for 1 minute to 300 minutes, 10 minutes to 150 minutes, 10 minutes to 90 minutes, 30 minutes to 90 minutes, or 40 minutes to 80 minutes. In this case, in the stepwise heat treatment, the temperature may be preferably raised in a range of 1 to 20° C./min during each stage movement. In addition, the heat treatment may be performed in a separate vacuum oven, an oven filled with an inert gas, or the like, but is not limited thereto. In addition, the application may be performed using an applicator to form a film on a support.

The polyamideimide film according to one embodiment has an effect of suppressing optical deterioration (cloudiness) even when the drying process is performed at a high temperature of about 140° C. at the time of the primary drying, such that it is possible to secure sufficient productivity for mass production.

In still another embodiment, there is provided a display device comprising the polyamideimide film provided in one embodiment. In addition, various types of molded articles may be manufactured using the polyamideimide film according to one embodiment. For example, the polyamideimide film may be applied to a protective film that may replace a printed wiring board, flexible circuit board, and cover glass comprising a protective film or an insulating film, and has a wide range of applications in various industrial fields, such as a window cover film and a flexible display panel.

The window cover film may be used as the outermost window substrate of a flexible display device. The flexible display device may be various image display devices such as a general liquid crystal display device, electro-luminescence display device, plasma display device, and field emission display device. As a display device comprising the window cover film provided in one embodiment has excellent display quality and implements a significant reduction in distortion caused by light, excellent visibility is provided, such that the user's eye fatigue may be minimized.

Hereinafter, Examples and Experimental Examples will be described in detail below. However, Examples and Experimental Examples to be described below are only to illustrate some embodiments, and the technology described in the present specification is not construed as being limited thereto.

<Measurement Methods>

1. Total Transmittance (Tt)

A total transmittance (%) of the film produced in each of Examples and Comparative Examples was measured in the entire 400 nm to 700 nm wavelength region using a transmittance meter (COH-5500, manufactured by Nippon Denshoku Industries Co., Ltd.) according to ASTM D1003 standard.

2. Haze

In order to measure transparency of the film produced in each of Examples and Comparative Examples, a value of a haze (%) was measured using a spectrophotometer (COH-5500, manufactured by Nippon Denshoku Industries Co., Ltd.) according to ASTM D1003 standard.

3. Yellow Index (YI)

A yellow index of the film produced in each of Examples and Comparative Examples was measured using a spectrophotometer (COH-5500, manufactured by Nippon Denshoku Industries Co., Ltd.) according to ASTM E313 standard.

4. Modulus

A modulus (GPa) of the polyamideimide film having a length of 50 mm and a width of 10 mm produced in each of Examples and Comparative Examples was measured under a condition in which the film was pulled at 25° C. and 50 mm/min using UTM 3365 manufactured by Instron Corporation according to ASTM D882 standard.

### Example 1

Under a nitrogen atmosphere, DMAc, TFMB, and AB-TFMB were put into a reactor, the mixture was sufficiently stirred, CBDA was put into the reactor, and the mixture was sufficiently stirred until dissolved. Then, BPAF and TPC were put into the reactor, and the mixture was stirred for 6 hours to be dissolved and reacted, thereby preparing a polyamic acid resin composition. At this time, a molar ratio of diamine monomers of TFMB:AB-TFMB was set to 50:50, and a molar ratio of the other monomers of TPC:BPAF:CBDA was set to 35:15:50. In addition, a solid content was adjusted to be 11.0 wt %, and the temperature of the reactor was maintained at 40° C.

Subsequently, pyridine and acetic anhydride were added to the solution at 2.5 times the moles of CBDA and BPAF, respectively, and the mixture was stirred at 60° C. for 6 hours. Thereafter, a solid obtained by precipitating the solution in an excessive amount of methanol and performing filtration was dried at 80° C. for 6 hours or longer to obtain a polyamideimide powder. 12 g of the obtained polyamideimide powder was dissolved in 88 g of DMAc, and solution casting was performed on a glass substrate using an applicator. A final viscosity of the solution was 25,000 cps when measured at 25° C. using a Brookfield viscometer. Thereafter, primary drying was performed using a convection oven at each of temperatures of 90° C. and 140° C. for 30 minutes, an additional heat treatment was performed under a nitrogen condition at a temperature of 280° C. for 1 hour to cool the resultant product to room temperature, and a film formed on the glass substrate was separated from the substrate, thereby producing a polyamideimide film.

### Examples 2 to 7

Polyamideimide films of Examples 2 to 7 were produced in the same manner as that of Example 1, except that the molar ratios of the monomers of TFMB:AB-TFMB and TPC:BPAF:CBDA were set as shown in Table 1.

### Example 8

A polyamideimide film was produced in the same manner as that of Example 1, except that under a nitrogen atmosphere, DMAc and AB-TFMB were put into a reactor, the mixture was sufficiently stirred, BPAF and TPC were put into the reactor, the mixture was stirred for 6 hours to be dissolved and reacted to prepare a polyamic resin composition, and at this time, the molar ratio of the monomers of TPC:BPAF was set to 55:45.

### Example 9

A polyamideimide film was produced in the same manner as that of Example 1, except that under a nitrogen atmosphere, DMAc and AB-TFMB were put into a reactor, the mixture was sufficiently stirred, CBDA was put into the reactor, and the mixture was sufficiently stirred until dissolved. Then, BPAF and TPC were put into the reactor, the mixture was stirred for 6 hours to be dissolved and reacted to prepare a polyamic acid resin composition, and at this time, a molar ratio of the monomers of TPC:BPAF:CBDA was set to 55:15:30.

### Comparative Examples 1 to 4

Polyamideimide films of Comparative Examples 1 to 4 were produced in the same manner as that of Example 1, except that 6FDA was used as the aromatic dianhydride instead of BPAF, and the molar ratios of the monomers of TFMB:AB-TFMB and TPC:6FDA:CBDA were set as shown in Table 1.

### <Experimental Example 1> Evaluation of Film Formability

Thicknesses of the polyamideimide films produced in Examples and Comparative Examples were measured three times each with a thin film thickness meter (p-bite, manufactured by TESA SE). The average values thereof are shown in Table 1. As a result of the measurement, it was confirmed that in the cases of the polyamideimide films of Examples 1 to 9, a thickness sufficient to be used as a flexible window cover film was formed.

TABLE 1

| | | Composition (molar ratio) | | | | | |
|---|---|---|---|---|---|---|---|
| | | Diamine | | Aromatic diacid dichloride | | | |
| | | (Based on total of 100) | | and aromatic dianhydride | | | |
| | Thickness | | AB- | (Based on total of 100) | | | |
| Classification | (μm) | TFMB | TFMB | TPC | BPAF | 6FDA | CBDA |
| Example 1 | 50 | 50 | 50 | 35 | 15 | — | 50 |
| Example 2 | 50 | 50 | 50 | 35 | 20 | — | 45 |
| Example 3 | 50 | 60 | 40 | 35 | 20 | — | 45 |
| Example 4 | 50 | 60 | 40 | 40 | 20 | — | 40 |
| Example 5 | 50 | 65 | 35 | 45 | 15 | — | 40 |
| Example 6 | 51 | 70 | 30 | 50 | 15 | — | 35 |
| Example 7 | 48 | 77 | 23 | 55 | 15 | — | 30 |
| Example 8 | 50 | — | 100 | 55 | 45 | — | — |
| Example 9 | 50 | — | 100 | 55 | 15 | — | 30 |
| Comparative | 49 | 50 | 50 | 35 | — | 15 | 50 |
| Example 1 | | | | | | | |
| Comparative | 48 | 50 | 50 | 35 | — | 20 | 45 |
| Example 2 | | | | | | | |
| Comparative | 47 | 70 | 30 | 50 | — | 15 | 35 |
| Example 3 | | | | | | | |
| Comparative | 52 | 77 | 23 | 55 | — | 15 | 30 |
| Example 4 | | | | | | | |

### <Experimental Example 2> Analysis of Physical Properties of Polyamideimide Film

In order to confirm the physical properties of the polyamideimide films produced in Examples and Comparative Examples according to the primary drying temperatures (90° C. and 140° C.), the transmittance, the haze, the yellow index, and the modulus were measured according to the measurement methods. The results thereof are shown in Table 2.

TABLE 2

| | Drying | Physical properties of film | | | |
|---|---|---|---|---|---|
| | temperature | Tt | Haze | Yellow | Modulus |
| Classification | (° C.) | (%) | (%) | index | (GPa) |
| Example 1 | 90 | 88.5 | 1.0 | 5.6 | 9.4 |
| | 140 | 88.3 | 1.1 | 6.5 | 9.5 |
| Example 2 | 90 | 89.5 | 0.4 | 3.5 | 8.4 |
| | 140 | 89.0 | 0.8 | 5.0 | 7.8 |
| Example 3 | 90 | 89.7 | 0.4 | 3.9 | 7.9 |
| | 140 | 89.5 | 0.5 | 3.9 | 7.6 |
| Example 4 | 90 | 89.7 | 0.3 | 3.3 | 7.8 |
| | 140 | 89.7 | 0.4 | 3.2 | 7.8 |
| Example 5 | 90 | 89.4 | 0.8 | 3.9 | 8.3 |
| | 140 | 89.0 | 0.9 | 5.1 | 7.9 |
| Example 6 | 90 | 89.7 | 0.3 | 3.2 | 8.1 |
| | 140 | 89.6 | 0.8 | 3.4 | 7.7 |
| Example 7 | 90 | 90.2 | 0.3 | 2.8 | 7.2 |
| | 140 | 90.0 | 0.3 | 3.0 | 7.0 |
| Comparative | 90 | 88.7 | 0.9 | 4.9 | 9.2 |
| Example 1 | 140 | 85.2 | 8.2 | 14.8 | 8.7 |
| Comparative | 90 | 88.9 | 0.6 | 5.2 | 8.6 |
| Example 2 | 140 | 88.0 | 2.1 | 7.6 | 8.2 |
| Comparative | 90 | 89.1 | 0.8 | 3.9 | 8.3 |
| Example 3 | 140 | 88.9 | 2.0 | 5.8 | 7.8 |
| Comparative | 90 | 89.5 | 0.6 | 4.2 | 7.7 |
| Example 4 | 140 | 88.9 | 2.1 | 6.0 | 7.1 |

Referring to Table 2, it could be appreciated that in the cases of the polyamideimide films according to Examples obtained using the composition containing a polyamideimide precursor and/or polyamideimide, a thickness sufficient to be used as a flexible window cover film was formed, not only when the drying process was performed at a temperature of 90° C., but also when the drying process was performed at a high temperature of 140° C., the total transmittance (Tt) was 88% or more, which was excellent, and the cloudiness at a high temperature was effectively suppressed. In addition, the haze was 1.5% or less, which showed that the transparency was significantly excellent, and the yellow index at 140° C. was 7 or less. That is, it was confirmed that in a case where the film according to each of Examples obtained using the composition containing a polyamideimide precursor and/or polyamideimide was compared with the film dried at 90° C., even when the film was dried at 140° C., there was no significant difference in optical and mechanical properties, a sufficient thickness was formed, and excellent optical and mechanical properties were maintained, and thus, the film was suitably used as a flexible window cover film.

On the other hand, in the cases of Comparative Examples 1 to 4 in which a drying process was performed at a high temperature of 140° C., it could be confirmed that the transmittance was 89% or less, which was lower than those of the films of Examples, and the haze was 2.0 or more, which showed that the transparency was significantly low, and the yellow index was 6 or more except for Comparative Example 3, which showed that the deterioration of the optical properties was accelerated.

As set forth above, the composition containing a polyamideimide precursor and/or polyamideimide according to one embodiment and the polyamideimide film obtained using the same may be applied to a display device because the composition and the polyamideimide film have excellent mechanical and optical properties.

Hereinabove, one embodiment has been described in detail through Examples and Comparative Examples, but the scope of one embodiment is not limited to a specific embodiment, and should be interpreted according to the appended claims.

1. A composition comprising:
a polyamideimide precursor or polyamideimide having a unit derived from an aromatic diamine, a unit derived from an aromatic dianhydride, and a unit derived from an aromatic diacid dichloride; and
a solvent,
wherein the aromatic diamine comprises a compound represented by the following Chemical Formula 1 (AB-TFMB), the aromatic dianhydride comprises 9,9-bis(3,4-dicarboxyphenyl)fluorene dianhydride (BPAF), and the aromatic diacid dichloride comprises terephthaloyl dichloride (TPC):

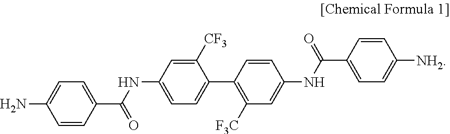 [SMILES: Nc1ccc(C(=O)Nc2ccc(-c3ccc(NC(=O)c4ccc(N)cc4)cc3C(F)(F)F)c(C(F)(F)F)c2)cc1]

2. The composition of claim 1, wherein the aromatic diamine further comprises a second aromatic diamine different from the compound represented by Chemical Formula 1.

3. The composition of claim 2, wherein the second aromatic diamine contains an aromatic ring substituted with a trifluoroalkyl group.

4. The composition of claim 2, wherein the second aromatic diamine is 2,2′-bis(trifluoromethyl)-benzidine (TFMB).

5. The composition of claim 1, wherein the polyamideimide precursor or polyamideimide further has a unit derived from an aliphatic ring-containing dianhydride.

6. The composition of claim 5, wherein the aliphatic ring-containing dianhydride comprises cyclobutane-1,2,3,4-tetracarboxylic dianhydride (CBDA).

7. The composition of claim 5, wherein a ratio of the number of moles of the unit derived from the aliphatic ring-containing dianhydride to the total number of moles of the unit derived from the aromatic diacid dichloride and the unit derived from the aromatic dianhydride is 95:5 to 10:90.

8. A polyamideimide film comprising a cured product of the composition of claim 1.

9. The polyamideimide film of claim 8, wherein the polyamideimide film has a total transmittance of 87.0% or more when measured at 400 nm to 700 nm according to ASTM D1003 standard.

10. The polyamideimide film of claim 8, wherein the polyamideimide film has a haze of 2.0% or less when measured according to ASTM D1003 standard.

11. The polyamideimide film of claim 8, wherein the polyamideimide film has a yellow index of 7.0 or less when measured according to ASTM E313 standard.

12. The polyamideimide film of claim 8, wherein a thickness of the polyamideimide film is 1 μm to 500 μm.

13. A display device comprising the polyamideimide film of claim 8.
